(19) United States
(12) Patent Application Publication
(10) Pub. No.: US 2026/0224220 A1
(43) Pub. Date: Aug. 6, 2026

(54) OCCLUSION PLUG

(71) Applicant: TOKAI MEDICAL PRODUCTS, INC., Aichi (JP)

(72) Inventors: Hironari KATO, Aichi (JP); Shingo YAMADA, Aichi (JP)

(73) Assignee: TOKAI MEDICAL PRODUCTS, INC., Aichi (JP)

(21) Appl. No.: 19/132,083

(22) PCT Filed: Nov. 21, 2023
(86) PCT No.: PCT/JP2023/041826

§ 371 Date: May 22, 2025

(30) Foreign Application Priority Data
Nov. 24, 2022 (JP) 2022-187572

(51) Int. Cl. A61B 17/12

(52) U.S. Cl. CPC A61B 17/12109; A61B 17/12172; A61B 2017/1205

(57) ABSTRACT

The purpose of the present invention is to provide an occlusion plug which makes it possible to secure occlusion performance by making the meshes of the plug finer and also makes it possible to secure the diameter reducing properties of the occlusion plug. For achieving the purpose, this occlusion plug (100) comprises a braided structure formed from wires (11), is formed into a narrower shape so as to be inserted into a delivery catheter or a sheath when the occlusion plug (100) is in a drawn state, and is inflated and expanded in the radiation direction when the occlusion plug (100) is in the opened state to press the inside of a blood vessel so as to place the occlusion plug (100) inside the blood vessel, thereby occluding the blood vessel. The occlusion plug (100) is characterized in that a single bundle of a plurality of wires (11) is braided and inweaved.

### TECHNICAL FIELD

The present invention relates to an occlusion plug.

### BACKGROUND ART

As a device for blood vessel occlusion that is percutaneously indwelled in arteriovenous vessels and blocks blood flow, there is an occlusion plug having a wire braided structure (Patent Literature 1). The occlusion plug having such a wire braided structure is stretched in the axial direction to deform the mesh shape and reduce the outer diameter. By being thinned by the diameter reduction, it is possible to advance in a blood vessel through an indwelling catheter. After the advancement, the stretch is released at the arrival point of a treatment site to expand the outer diameter, and an expansion force is applied to the blood vessel wall, so that the indwelling is performed without positional displacement. In the indwelling state, the finer the meshes, the smaller the gaps, the higher the occlusion capacity, and the blood flow can be stopped. As described above, in the occlusion plug, two elements of a diameter reduction function and an occlusion capacity are indispensable, but if the meshes are made too fine in order to enhance the occlusion capacity, the deformation of the mesh shape at the time of stretching becomes difficult, and the diameter reduction amount decreases. On the other hand, when the meshes are coarse, smooth deformation is possible, but the occlusion capacity is deteriorated. Therefore, the occlusion plug is preferably one having as fine meshes as possible under the condition satisfying the diameter reduction that allows the indwelling catheter to be inserted. In order to satisfy such conditions as much as possible, in the current market, products that compensate for the condition of fineness of meshes by laminating two or three layers of braided wires in multiple layers are widely used (Patent Literature 2).

A multi-layer occlusion plug has a higher occlusion capacity than that of a single-layer structure. However, compared to the single-layer, there are the problems that the multi-layer occlusion plug is generally harder, which can result in excessive pressure and adverse effects such as blood vessel damage, as well as increased resistance at the time of insertion of a delivery catheter, which can hinder deformation operation also at the time of indwelling the occlusion plug, and can adversely affect treatment.

### CITATIONS LIST

### Patent Literature

Patent Literature 1: WO2014/110589
Patent Literature 2: JP 2005-261951 A

### SUMMARY OF INVENTION

### Technical Problems

Therefore, an object of the present invention is to provide an occlusion plug capable of securing an occlusion capacity by making meshes finer and securing a diameter reduction property of the occlusion plug.

### Solutions to Problems

An occlusion plug according to the present invention has a wire braided structure, is formed to be thin so as to be insertable into a delivery catheter or a sheath in a stretched state, and is expanded and developed in a radial direction in an open state to press and indwell in a blood vessel to embolize the blood vessel, in which
the occlusion plug is formed by braiding and weaving a plurality of wires regarded as one wire.

The occlusion plug according to the present invention is formed by braiding and weaving a plurality of wires regarded as one wire, so that it is possible to secure the occlusion capacity by making meshes finer and use a thin wire for each wire constituting the occlusion plug with respect to the wire constituting the occlusion plug braided with each normal wire because the plurality of wires is regarded as one wire. Therefore, the diameter reduction property of the occlusion plug can also be secured.

In the occlusion plug according to the present invention, the plurality of wires may be arranged side by side in parallel. By adopting such a configuration, even when the plurality of wires are braided and woven, it is possible to prevent a thick yarn from being formed by the plurality of wires and to obtain the occlusion plug having a high diameter reduction property.

In the occlusion plug according to the present invention, at least one of the plurality of wires may have a different wire diameter. By adopting such a configuration, the radial force can be increased without increasing the tension.

Furthermore, in the occlusion plug according to the present invention, the weaving may have a structure of any of plain weave, twill weave, and satin weave. By adopting such a woven structure, it is possible to secure the occlusion capacity by making meshes finer, and to secure the diameter reduction property of the occlusion plug.

Furthermore, in the occlusion plug according to the present invention, the occlusion plug may be formed in a disk shape when expanded and developed in an open state. By being formed in such a disk shape, it is possible to most expand from the stretched state.

Furthermore, in the occlusion plug according to the present invention, the occlusion plug may have a plurality of disk portions that are formed in a disk shape when expanded and developed in an open state and are arranged in parallel. By arranging the plurality of disk portions in parallel, the occlusion force can be improved.

Furthermore, in the occlusion plug according to the present invention, the occlusion plug may have three or more disk portions that are formed in a disk shape when expanded and developed in an open state and arranged in parallel, and a diameter of the disk portion other than the disk portions at both ends is formed to be smaller than diameters of the disk portions at both ends. As described above, since the length at the time of indwelling is reduced by mixing a disk having a small diameter, there is an effect that the number of options of situations in which the disk can be indwelled increases.

### BRIEF DESCRIPTION OF DRAWINGS

FIG. 1 is a side view illustrating an embodiment of an occlusion plug 100 according to the present invention.

FIG. 2 is a side view illustrating another embodiment of the occlusion plug 100 according to the present invention.

FIG. 3 is a schematic view illustrating a braiding state of the occlusion plug 100 according to the present invention.

FIG. 4 provides cross-sectional views of the occlusion plug 100 according to the present invention and Comparative Examples 1 and 2.

FIG. 5 provides cross-sectional views illustrating other embodiments of the occlusion plug 100 according to the present invention.

FIG. 6 is a view illustrating a state in which the occlusion plug 100 according to the present invention is inserted into a delivery catheter.

FIG. 7 is a view illustrating a state in which the occlusion plug 100 according to the present invention is indwelled in a blood vessel.

FIG. 8 is a diagram showing a test result of the occlusion plug 100 according to the present invention.

FIG. 9 is a view showing a test method of a water flow resistance test of the occlusion plug 100 according to the present invention.

### DESCRIPTION OF EMBODIMENTS

Hereinafter, embodiments of the present invention will be described with reference to the drawings, but the present invention is not limited thereto.

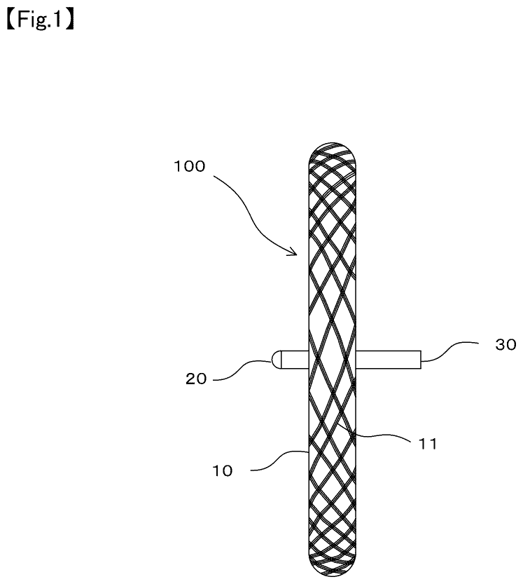

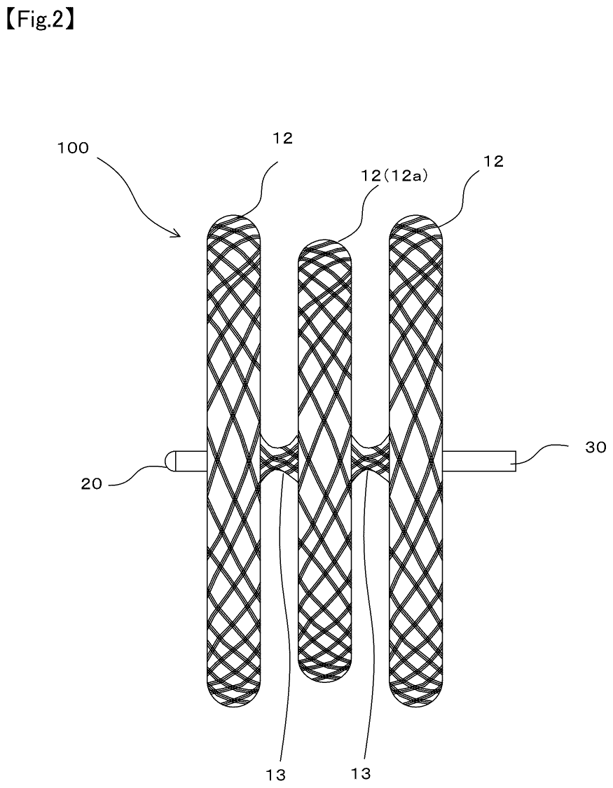

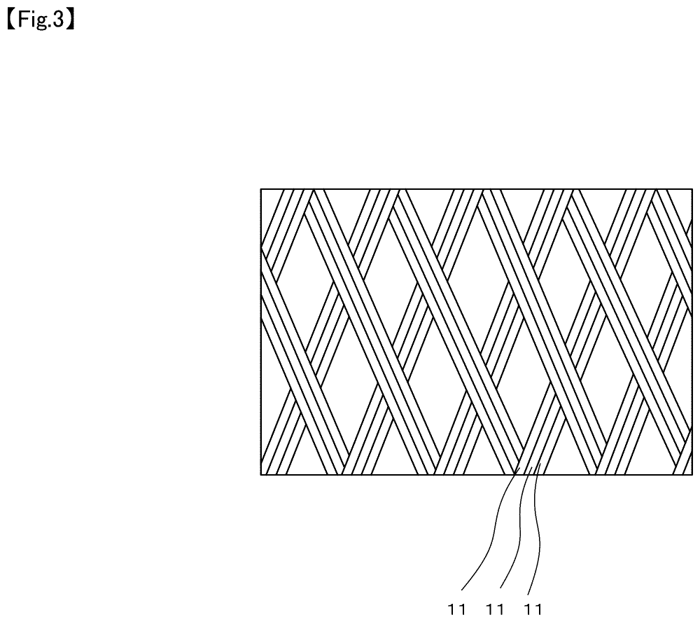

FIG. 1 is a diagram schematically illustrating an occlusion plug 100 according to the present invention. FIG. 2 is a diagram illustrating another embodiment of the occlusion plug 100. FIG. 3 is a schematic view illustrating a mesh structure of the occlusion plug 100.

The occlusion plug 100 according to the present invention includes a plug main body 10 that is expandable to a diameter equal to or larger than an inner diameter of a blood vessel, a distal end portion 20 formed at a distal end of the plug main body 10, and a detachment portion 30 connected to a pusher wire 40 to be operated when the occlusion plug 100 is indwelled.

The distal end portion 20 is a member that fixes the distal end side of a wire 11 constituting the mesh or the weave of the plug main body 10 in a bundled state. This distal end portion 20 may use a radiopaque material to improve visibility under radioscopy.

The plug main body 10 is a portion having a function of expanding in the radial direction and indwelling in the blood vessel to block the blood flow in the blood vessel when opened from a guiding catheter 50 in the blood vessel. The plug main body 10 has a braided or woven structure using a wire. As illustrated in FIG. 3, the braided structure or the woven structure has a braided or woven structure in which 2 to 10 wires 11 are regarded as one braided wire (in FIG. 3, three wires arranged side by side are regarded as one wire). As the woven structure at this time, any structure of plain weave, twill weave, and satin weave can be adopted. Since the wire 11 to be used is consisted of a plurality of wires that are regarded as one wire, a thin wire can be used when the same pressure against the blood vessel wall is to be applied, compared to a normal plug main body braided with one wire. Preferably, a wire having a thickness of 0.020 mm to 0.100 mm is used. The external shape of the occlusion plug 100 is not particularly limited as long as it can be reduced in diameter to such an extent that it can be inserted into the guiding catheter 50 in the stretched state, and can be expanded to have a diameter larger than the inner diameter of the blood vessel at the indwelling position. Preferably, as illustrated in FIG. 1, it is preferable to be produced so as to have a disk shape in the expanded state (open state). By forming in such a disk shape, it is possible to expand from the stretched state. In addition, by reducing the thickness (width in the longitudinal direction) of the disk shape, the length that can be placed at the time of indwelling is shortened, so that there is an effect that the number of options of situations in which the disk can be indwelled increases. Furthermore, as illustrated in FIG. 2, two or more disk portions 12 having such a disk shape (in FIG. 2, three are arranged in parallel) may be formed in parallel via constricted portions 13. At this time, in the specification in which three or more of the disk portions 12 are connected and arranged in parallel, the pressure force applied to the blood vessel by a disk portion 12a other than the disk portions 12 at both ends can be set lower than those by the disk portions 12 arranged at both ends, so that a disk having a smaller diameter than the disk portions 12 arranged at both ends can be mixed in. As described above, since the length at the time of indwelling is reduced by mixing a disk having a small diameter, there is an effect that the number of options of situations in which the disk can be indwelled increases.

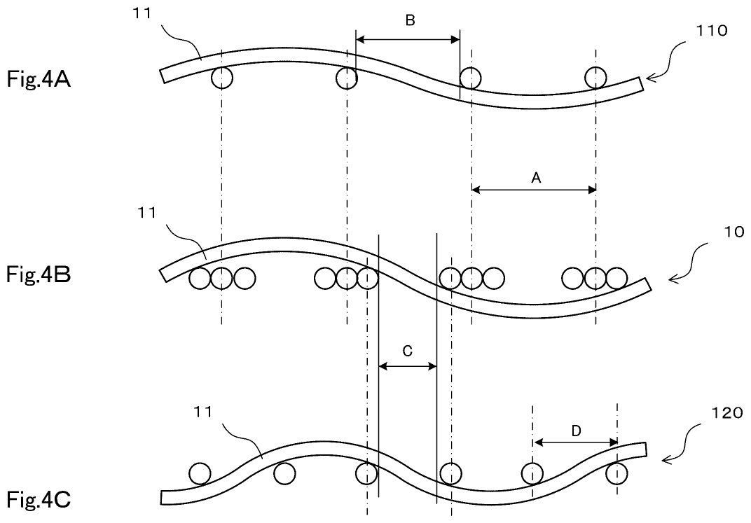

Thus, the following effects are obtained by braiding the plurality of wires 11 in parallel. Describing with a 2-over-2-under twill weave illustrated in FIG. 3, when the cross section (FIG. 4B) of the plug main body 10 according to the present invention is compared with the cross section (FIG. 4A) of a plug main body 110 according to Comparative Example 1 made by single wire braiding with the same braiding pitch A, in the plug main body 110 of Comparative Example 1, since the curvature of the wire remains small, the tension applied between the wires does not change and flexibility is maintained, but since a gap B becomes large, the occlusion capacity of the blood flow decreases. On the other hand, as compared with the cross section (FIG. 4C) of a plug main body 120 according to Comparative Example 2 made by single wire braiding with a gap C having the same width, the wire of the plug main body 120 according to Comparative Example 2 has a shorter braiding pitch D, a larger curvature of the wire, and a higher tension between the wires. Therefore, there is a disadvantage that flexibility of the plug main body is lost, elasticity is reduced, and the plug main body is easily twisted. In contrast, in the plug main body 10 according to the present invention, it is possible to keep the tension of the wire 11 low while keeping the gap small, and it is possible to provide the flexible occlusion plug 100 having a high occlusion capacity of the blood flow.

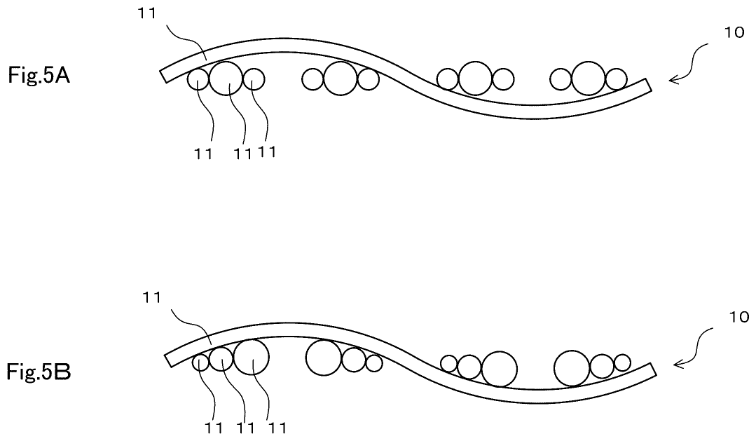

Modifications of the braided or woven structure of the plug main body 10 are illustrated in FIG. 5. In these, when the plurality of wires 11 are braided in parallel, wires of different thicknesses are used. By adopting such a structure, the radial force (reaction force when the diameter is reduced) can be increased without increasing the tension. For example, FIG. 5A illustrates a braided or woven structure using a wire having a larger diameter than the remaining two wires in the middle of the three parallel wires. FIG. 5B illustrates a braided or woven structure in which three wires of all different diameters are arranged in ascending order of diameter. By adopting such a structure, the radial force can be increased without increasing the tension. In addition, as illustrated in FIG. 5B, the contact rate of the intersecting wires can be increased.

The detachment portion 30 is formed so as to fix the hand side of the wire 11 constituting the mesh of the plug main body 10 in a bundled state, and to connect or detach the plug main body 10 and the pusher wire 40 when delivering the occlusion plug to an indwelling region. Examples of the structure of the detachment portion 30 include, but are not limited to, a configuration in which a screw groove is formed and the plug main body is detachable by rotating the pusher wire 40.

The occlusion plug 100 thus produced is used as follows. First, a blood vessel diameter in the indwelling region is measured, and the occlusion plug 100 having a diameter larger than the blood vessel diameter by about 15% to 70% in the expanded state is prepared. A sheath or guiding catheter 50 compatible with the selected occlusion plug 100 is prepared, a Y-connector is attached thereto, the occlusion plug 100 is inserted into a blood vessel via a guide wire and delivered to an indwelling position, and the guide wire is removed.

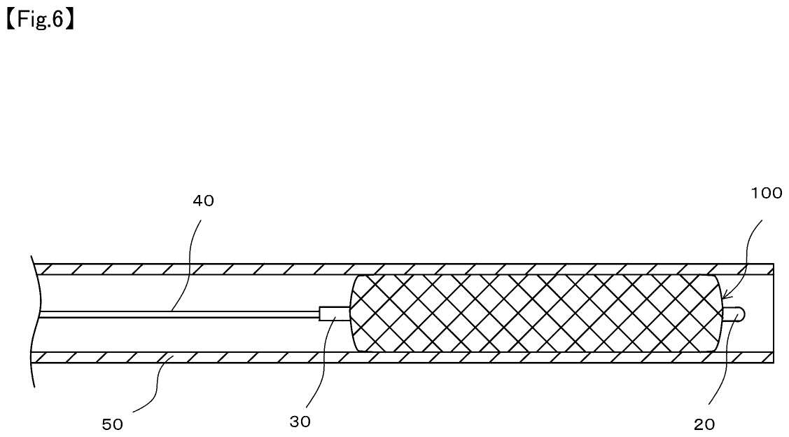

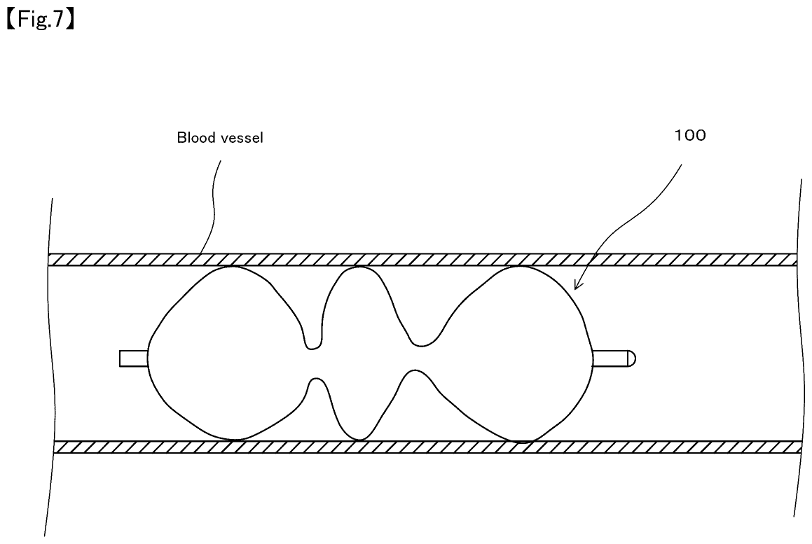

Thereafter, as illustrated in FIG. 6, the occlusion plug 100 is advanced to the distal end portion of the sheath or the guiding catheter 50 by the pusher wire 40. Confirmation contrast imaging is performed through the sheath or guiding catheter 50 to confirm that the occlusion plug 100 is in the indwelling position in the sheath or guiding catheter 50, and the plug is deployed by holding the position of the occlusion plug 100 and pulling the sheath or guiding catheter 50. Then, the pusher wire 40 and the sheath or the guiding catheter 50 are removed together to the outside of the body. As a result, as illustrated in FIG. 7, the occlusion plug is indwelled so as to block the inside of the blood vessel.

According to the occlusion plug 100 of the present invention, since the occlusion plug 100 has a woven structure in which about 2 to 10 wires are regarded as one braided wire, the occlusion force increases because the meshes are finely formed as compared with the case of braiding with one wire. On the other hand, by regarding the plurality of wires as one braided wire, it is possible to use a small-diameter wire in one layer. Therefore, it is possible to deform the wire in accordance with the shape of the blood vessel and the status of the indwelling position without resulting in excessive pressure unlike the two-layer structure, and it is possible to smoothly insert the wire in accordance with the meandering of the blood vessel at the time of delivery. In addition to the braided structure of the plurality of wires, by adopting a form that has a disk shape in the expanded state, a high expansion force can be secured, the possibility that the indwelling position is shifted due to the blood pressure can be reduced, and the total length of the occlusion plug can be shortened, so that there is an effect that a situation in which the occlusion plug can be indwelled is widened.

### EXAMPLES

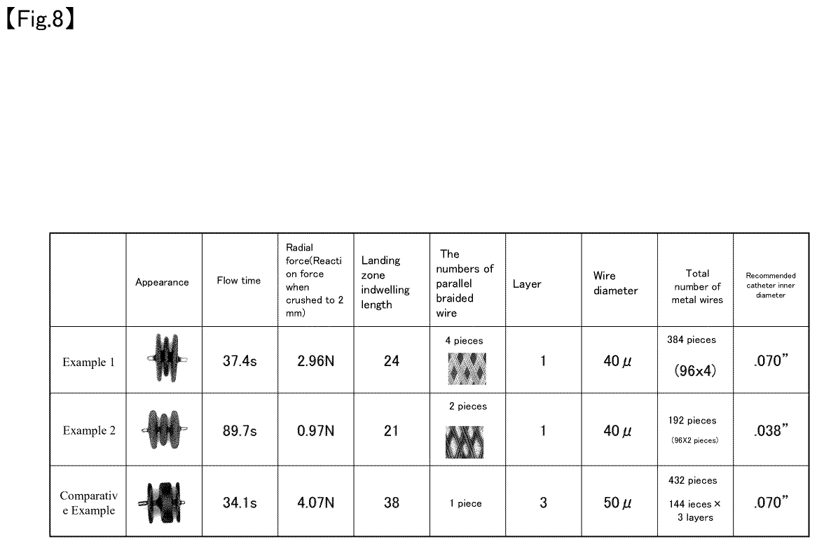

As Example 1 of the occlusion plug 100 according to the present invention, as illustrated in FIG. 8, an occlusion plug was produced using a braided wire with a wire diameter of 40 μm, woven in one layer with four parallel wires, with three disk portions arranged in parallel, and with the central disk having a smaller diameter. As Example 2, an occlusion plug was produced using a braided wire with a wire diameter of 40 μm, woven in one layer with two parallel wires, with three disk portions arranged in parallel, and with the central disk having a smaller diameter. As Comparative Example, an occlusion plug was produced using a braided wire with a wire diameter of 50 μm, woven in three layers with one parallel wire, with two disk portions, and a member between them having a cylindrical shape.

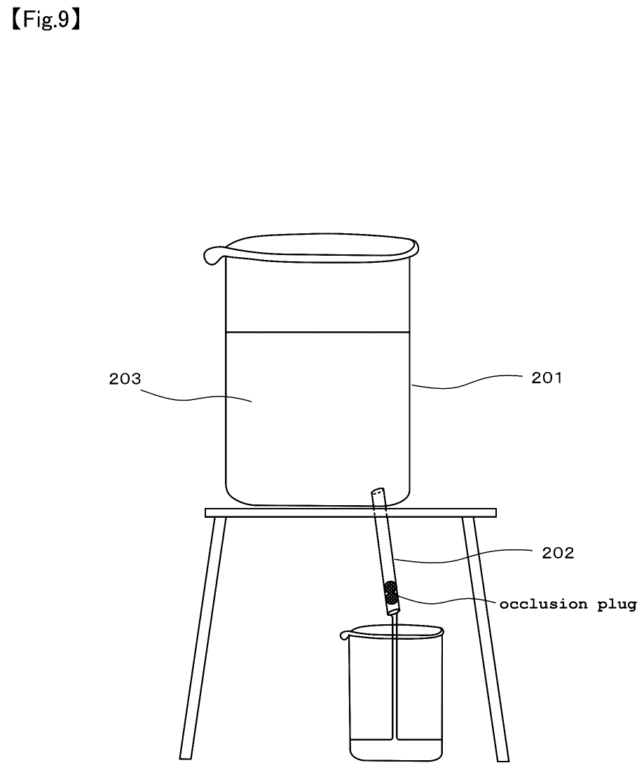

Using these Examples and Comparative Example, a water flow resistance test was performed to compare occlusion performance. In the water flow resistance test, as illustrated in FIG. 9, a silicon tube 202 (simulated blood vessel) having a diameter of 8 mm was connected to the bottom of a beaker 201, an occlusion plug of each of Examples 1 and 2 and Comparative Example having a diameter of 12 mm was indwelled in the silicon tube, and the time until 200 ml of a glycerin solution 203 (simulated blood) was completely flowed was measured. As shown in FIG. 8, the result of Example 1 was 37.4 seconds and that of Example 2 was 89.7 seconds, whereas the result of Comparative Example 1 was 34.1 seconds. As a result, it can be seen that the occlusion performance is higher in Examples according to the present invention. The radial force is 2.96 N in Example 1 and 0.97 N in Example 2, whereas the radial force is 4.07 N in Comparative Example 1, which indicates that the delivery performance is higher than that of Comparative Example. Furthermore, the landing zone indwelling length is 24 mm in Example 1 and 21 mm in Example 2, whereas it is 38 mm in Comparative Example 1, and it can be seen that the indwelling length in Comparative Example 1 is long. This is an advantageous effect resulting from that the diameter of the central disk portion of this Example is short.

10 . . . plug main body, 11 . . . wire, 12 . . . disk portion, 13 . . . constricted portion, 20 . . . distal end portion, 30 . . . detachment portion, 40 . . . pusher wire, 50 . . . guiding catheter, 100 . . . occlusion plug

1. An occlusion plug that has a wire braided structure, is formed to be thin so as to be insertable into a delivery catheter or a sheath in a stretched state, and is expanded and developed in a radial direction in an open state to press and indwell in a blood vessel to embolize the blood vessel, wherein
the occlusion plug is formed by braiding and weaving a plurality of wires regarded as one wire.

2. The occlusion plug according to claim 1, wherein the plurality of wires are arranged side by side in parallel.

3. The occlusion plug according to claim 1, wherein at least one of the plurality of wires has a different wire diameter.

4. The occlusion plug according to claim 1, wherein the weaving has a structure of any of plain weave, twill weave, and satin weave.

5. The occlusion plug according to claim 1, wherein the occlusion plug is formed in a disk shape when expanded and developed in an open state.

6. The occlusion plug according to claim 1, wherein the occlusion plug has a plurality of disk portions that are formed in a disk shape when expanded and developed in an open state and are arranged in parallel.

7. The occlusion plug according to claim 1, wherein the occlusion plug has three or more disk portions that are formed in a disk shape when expanded and developed in an open state and arranged in parallel, and a diameter of the disk portion other than the disk portions at both ends is formed to be smaller than diameters of the disk portions at both ends.
